(19) United States
(12) Patent Application Publication
(10) Pub. No.: US 2016/0157719 A1
(43) Pub. Date: Jun. 9, 2016

(54) MULTIFUNCTION BIOSENSOR SYSTEM

(71) Applicant: Donald SPECTOR, New York, NY (US)

(72) Inventor: Donald SPECTOR, New York, NY (US)

(21) Appl. No.: 14/826,364

(22) Filed: Aug. 14, 2015

Related U.S. Application Data
(60) Provisional application No. 62/087,036, filed on Dec. 3, 2014.

(51) Int. Cl. A61B 5/00; A61B 5/0402; A61B 5/0205

(52) U.S. Cl. CPC A61B 5/0031; A61B 5/742; A61B 5/0205; A61B 5/0402; A61B 5/0024; A61B 5/7405; A61B 5/746; A61B 2019/448

(57) ABSTRACT

A system for monitoring the health of an individual by providing biometric sensors attached to or inside the skin of a patient, along with a transmitter connected to the sensors for transmitting data from the sensors to a central monitoring station via a receiver located near the sensors. The sensor system is connected to an RFID tag, which communicates with an RFID reader in a remote location. The RFID tag transmits data from the sensors to the reader through the antenna, and the data is then analyzed by a microprocessor and an alert is communicated to a central monitoring station if the data from the sensors exceeds a preset threshold limit.

### CROSS REFERENCE TO RELATED APPLICATIONS

Applicant claims priority under 35 U.S.C. §119(e) of U.S. Provisional Patent Application Ser. No. 62/087,036 filed Dec. 3, 2014, the disclosure of which is incorporated by reference.

### BACKGROUND OF THE INVENTION

1. Field of the Invention

This invention relates to a biometric sensor system that utilizes biosensors that are inserted into the skin of a patient. In particular the invention relates to a biometric sensor device can sense a wearer's health status and communicate the status to a central monitoring station.

2. The Prior Art

It is known to attach a sensor to a patient to monitor the patient's health and communicate the data collected by the monitor to a central station. U.S. Pat. No. 8,823,512 to Spector describes a patient monitoring system where the biometric sensor communicates with a receiver which transmits the information along with an alarm code to a central monitoring center. The receiver is often the user's cell phone, which communicates with a sensor worn by the user. While this is a convenient way to monitor the health of a person, it would be useful if the sensor was attached to the patient so that it cannot be removed. In addition, it would be useful if the device was programmable via external communications so that the device can change the physiological characteristics that are being measured by directing different sensors to be activated.

### SUMMARY OF THE INVENTION

The invention provides a system for monitoring the health of an individual by providing biometric sensors attached to or inside the skin of a patient, along with a transmitter connected to the sensors for transmitting data from the sensors to a central monitoring station via a receiver located near the sensors.

The sensor system is connected to an RFID tag, which communicates with an RFID reader in a remote location. The RFID tag is a transmitter comprised of a microchip connected to an antenna, that is interrogated by the reader or transmits directly to the reader when in range of the tag. The RFID tag has an independent power source to transmit data from the sensors to the RFID reader. The RFID tag transmits data from the sensors to the reader through the antenna, and the data is then analyzed and communicated to a central monitoring station. Alternatively, other types of transmitters and receivers could be used with the sensor system according to the invention.

The RFID reader is typically disposed in a portable monitoring unit such as a smartphone that has a microprocessor and data storage capability. The microprocessor receives data from the sensors and compares the data to stored reference data. If the received data exceeds a predetermined threshold of the reference data, the microprocessor then communicates an alarm status to the central monitoring station, via a transmitter using cellular data, Wifi or any other suitable means. The smartphone itself can also issue a visual or audible alarm. One or more central monitoring stations can be programmed to receive signals from the transmitter, and the microprocessor is programmed to cause the transmitter in the smartphone continuously transmit the emergency alert signal until it is picked up by one of the monitoring stations. The monitoring stations can be a server of a health care system, a mobile phone of a relative or friend, or any suitable computer having the capability of receiving a remote signal. The monitoring stations each have a receiver, a processor and a display.

Along with the alert, the mobile monitoring unit sends the sensor data to the monitoring stations, so that healthcare professionals can see the patient's status at the stations. The sensor data is not sent until an alert level has been reached, so that the monitoring stations are not bombarded with excess data from various sources during non-alert periods. The alert and sensor data are displayed on a display connected to the receiver and processor of the monitoring station. The alert could also be communicated audibly. The processor at the monitoring station is configured to encrypt data received from the transmitter and store the data in a database connected to the processor for future use. The alert is sent continuously from the smartphone to the monitoring station until the alert is acknowledges by the monitoring station.

The microprocessor connected to the RFID reader can be programmed to receive data from all sensors simultaneously, or can turn on or off different sensors at different times. The sensors can be any type of sensor, including blood glucose, heart rate, blood pressure, eeg, GPS or any other type of sensor.

The sensor system can be in the form of a tattoo-like device that is applied to the user's skin within the epidermis, so that the sensors can sense the desired biological functions. The tattoo can be configured of graphene or any other suitable material. The sensor module is not implanted underneath the skin, but within the layers of the epidermis. Some of the sensors can penetrate below the epidermis if necessary to obtain the required data.

The microprocessor can also be programmed to trigger an alert if the RFID reader goes out of range of the RFID tag, or if the sensor component of the system is removed from the patient.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a diagram of a patient and the monitoring system according to the invention;

FIG. 2 is a cross-sectional view of the layers of an epidermis with the sensor component embedded therein;

FIG. 3 is a diagram of the system according to the invention in use; and

FIG. 4 shows a flow diagram of the monitoring steps according to the invention.

### DETAILED DESCRIPTION OF THE PREFERRED EMBODIMENT

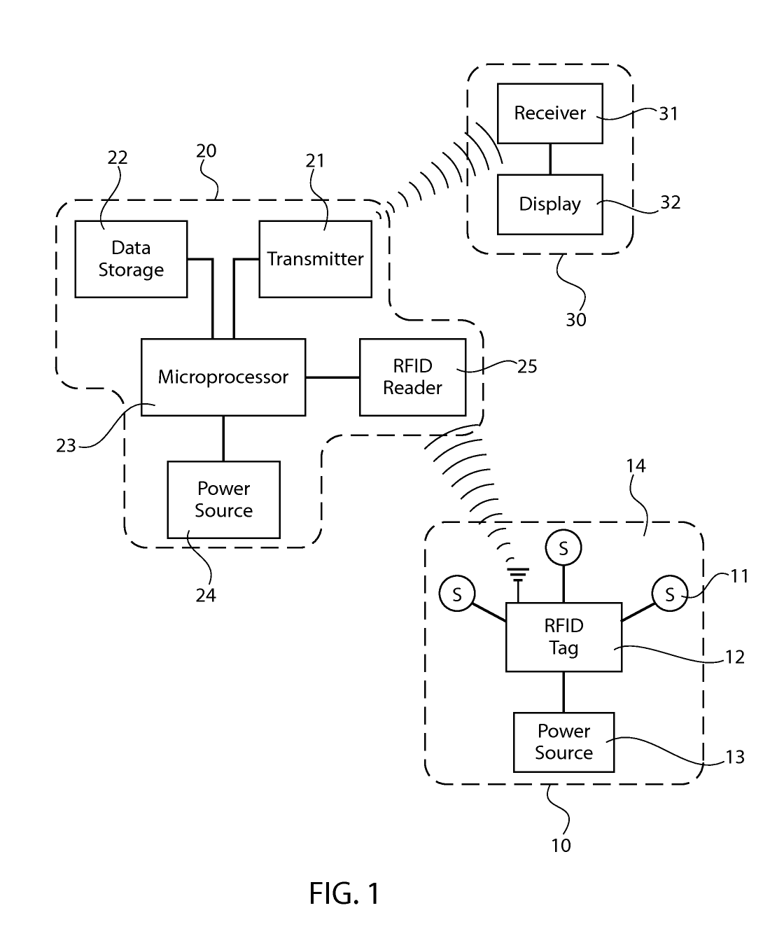

Referring now in detail to the drawings and, in particular, FIG. 1 shows a diagram of the system according to the invention. Sensor module 10 comprises an RFID tag 12 connected to a plurality of sensors 11 disposed on a substrate 14. Each of sensors 11 senses a different biological function, such as blood sugar, blood pressure, respiration rate, or any other biological function that is desired to be monitored. One of sensors 11 could also be a GPS sensor, which can determine the wearer's location at any time. A power source 13, which can be a battery, a temperature gradient power source or any other suitable power source, is connected to RFID tag 12 and allows tag 12 to transmit data directly to an RFID reader 25 disposed in a portable monitoring unit 20 located in a nearby location. Reader 25 is connected to a microprocessor 23, a power source 24 and a data storage 22. Data storage 22 stores data regarding the various biological functions that are being monitored, and stores a threshold level for each function. Data from sensors 11 is transmitted via RFID tag 12 to RFID reader 25. This data is then compared via microprocessor 23 with the threshold data stored in data storage 22, and if the sensed data exceeds the threshold, an alarm is transmitted via transmitter 21 to a remote receiver 31 in a central monitoring station 30. The alarm is then displayed on display 32. A display could also be connected to microprocessor 23 so that the alarm is directly displayed there too. The alarm could also be an audible alarm emanating directly from microprocessor 23 or via receiver 31. Transmitter 21 transmits via any suitable means, such as by Wifi, cellular transmission or any other suitable method for transmitting data over large distances. Portable monitoring unit 20 can be embodied as a mobile telephone or as any other small, portable unit that can be kept near the patient at all times.

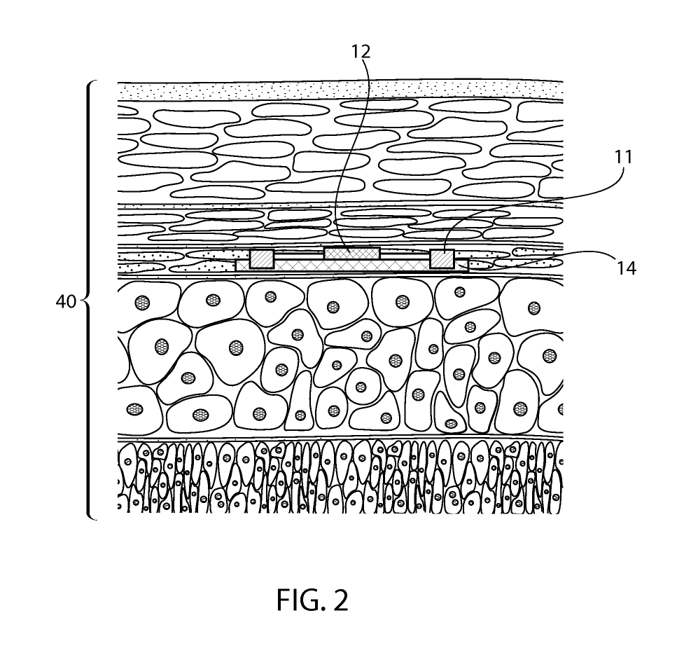

FIG. 2 shows sensor module 10 implanted within layers of a patient's epidermis 40. Sensors 11 extend through substrate 14 so that various biological functions can be sensed. Unlike traditional implants, sensor module 10 is not implanted deep within the body, but instead rests within the layers of the epidermis, so that it can be inserted and removed without large effort. To insert sensor module 10, a large needle can be used to inject the module within the layers of the epidermis. The module could be held in the needle in a rolled-up state and then unrolled after injection so that it rests flat in its place within the layers of the skin.

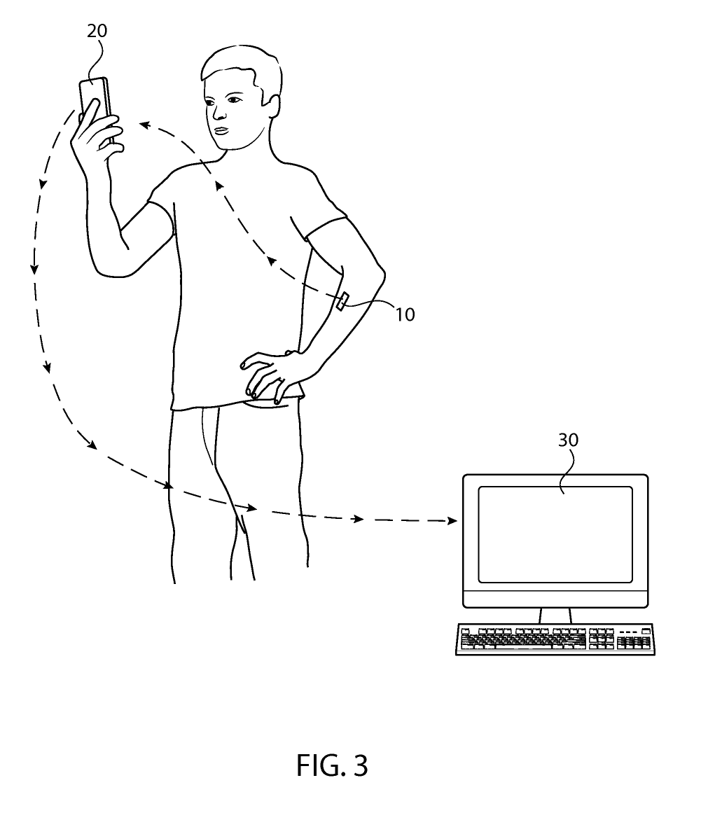

FIG. 3 shows the system according to the invention in use on a patient 50. Sensor module 10 is implanted within the layers of the epidermis of patient 50. Sensor module 10 communicates with portable monitoring unit 20, which is the form of a mobile telephone. Portable monitoring unit 20 is kept within a short range of sensor module 10 so that data from sensor module 10 can be communicated via RF to monitoring unit 20. If monitoring unit 20 goes out of range of sensor module 10, monitoring unit 20 sends an alert signal to central monitoring station 30, which comprises a remote computer where the status of sensor module 10 and the patient can be monitored. Alerts from portable monitoring unit 20 are communicated to central monitoring station 30 via Wifi, cellular data or any other suitable wireless communication means. The alerts could also be communicated to several stations at the same time, or to a server that maintains a website that can be accessed by any number of monitoring stations at once.

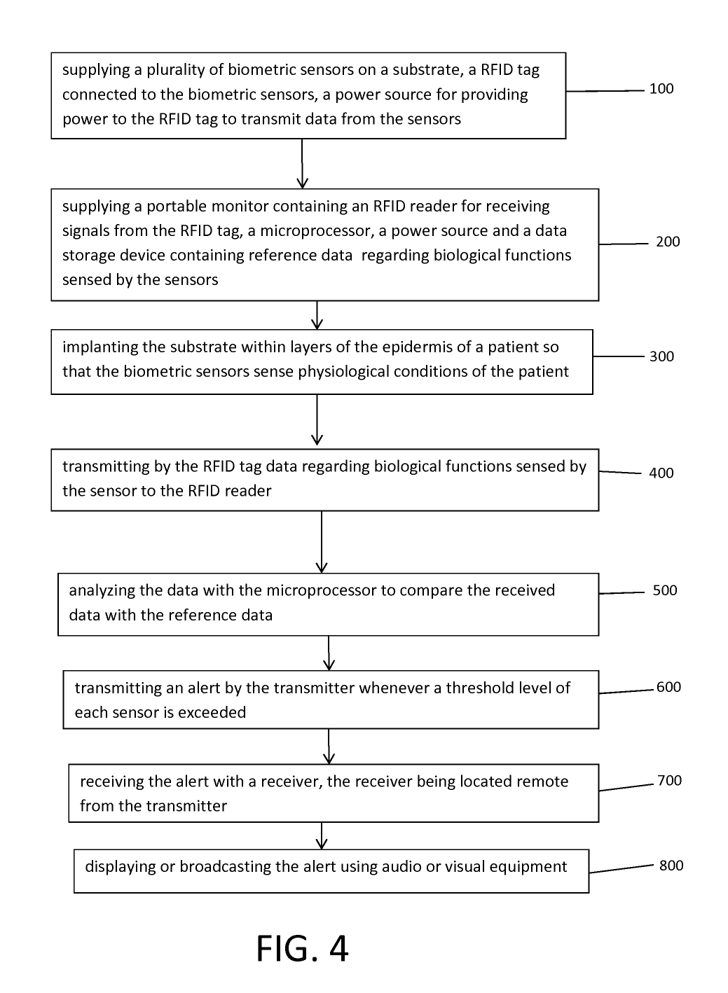

FIG. 4 shows a block diagram of the method steps according to the invention. In the method, with reference to the elements shown in FIGS. 1-3 as well, a plurality of biosensors 11, an RFID tag 12 connected to the biosensors 11, and a power source 24 for providing power to the RFID tag to transmit data from the sensors are all supplied on a substrate in step 100. In step 200, a portable monitor containing an RFID reader 25 for receiving signals from the RFID tag, a microprocessor 23, a power source 24 and a data storage device 22 containing reference data regarding biological functions sensed by the sensors 11 is supplied. Step 300 comprises implanting the substrate 14 within layers of the epidermis 40 of a patient so that the biometric sensors 11 sense physiological conditions of the patient. Step 400 comprises transmitting by the RFID tag 12 data regarding biological functions sensed by the sensor 11 to the RFID reader 25. Step 500 comprises analyzing the data with the microprocessor 23 to compare the received data with the reference data, and step 600 includes transmitting an alert by the transmitter 21 whenever a threshold level of each sensor 11 is exceeded. Step 700 comprises receiving the alert with a receiver 31, the receiver being located remote from the transmitter 21 and step 800 includes displaying or broadcasting the alert using audio or visual equipment such as a display 32.

Accordingly, while only a few embodiments of the present invention have been shown and described, it is obvious that many changes and modifications may be made thereunto without departing from the spirit and scope of the invention.

What is claimed is:

1. A patient monitoring system comprising:
a sensor module adapted to be placed within epidermal layers of a patient's skin, comprising:
a plurality of biometric sensors each being adapted to measure at least one biological function;
a transmitter configured to receive and transmit data from the biometric sensors;
a power source connected to the transmitter;
a portable monitor comprising:
a receiver disposed in a portable housing and being adapted to receive data from the at least one sensor;
a microprocessor connected to the receiver,
a data storage device connected to the microprocessor and containing reference data corresponding to biological functions sensed by the sensors and containing a threshold level for each biological function;
a second transmitter electrically connected to the microprocessor, the second transmitter being configured to transmit signals to a remote location;
a power source electrically connected to the RFID reader, microprocessor and second transmitter; and
a central monitoring station comprising:
at least one remote receiver configured for receiving alert signals from the transmitter;
a processor connected to each remote receiver, the processor being programmed to match signals from the second transmitter with a set of pre-programmed alert notifications; and
a display connected to the processor for displaying the alert notifications,
wherein the microprocessor is programmed to compare data received from the sensors with the threshold level stored in the data storage device and command the second transmitter to transmit an alert signal to the remote receiver when a threshold level has been exceeded.

2. The system according to claim 1, wherein the transmitter is an RFID tag and the receiver is an RFID reader.

3. The system according to claim 1, further comprising a speaker connected to the processor, wherein the processor is configured for sending an audible alert signal through the speaker when the threshold has been exceeded.

4. The system according to claim 1, wherein the portable monitor is embodied in a mobile telephone.

5. The system according to claim 1, wherein the remote receiver is embodied in a personal computer.

6. The system according to claim 1, wherein the biometric sensors are formed from graphene.

7. The system according to claim 1, wherein the biometric sensors are configured for measuring at least one of the following conditions: heart rate, blood oxygen level, blood glucose level, electrocardiogram, and respiration rate.

8. The system according to claim 1, wherein the processor in the remote receiver is configured to encrypt data received from the transmitter and store said data in a database connected to the processor.

9. The system according to claim 1, further comprising a GPS sensor connected to the transmitter.

10. The system according to claim 1, wherein the system is configured so that data from all sensors are sent to the microprocessor at the same time.

11. The system according to claim 1, wherein the microprocessor is configured to turn on and off the sensors so that only data from selected sensors is transmitted to the remote receiver.

12. A method for monitoring the health of a patient comprising:
supplying a plurality of biometric sensors on a substrate, a transmitter connected to the biometric sensors, a power source for providing power to the transmitter to transmit data from the sensors;
supplying a portable monitor containing a receiver for receiving signals from the transmitter, a microprocessor, a power source, a second transmitter and a data storage device containing reference data regarding biological functions sensed by the sensors;
implanting the substrate within layers of the epidermis of a patient so that the biometric sensors sense physiological conditions of the patient;
transmitting by the transmitter data regarding biological functions sensed by the sensor to the receiver;
analyzing the data with the microprocessor to compare the received data with the reference data;
transmitting an alert by the second transmitter whenever a threshold level of each sensor is exceeded;
receiving the alert with a remote receiver, the remote receiver being located remote from the transmitter; and
displaying or broadcasting the alert using audio or visual equipment.

13. The method according to claim 12, further comprising programming the microprocessor so that alerts from only selected sensors are transmitted to the receiver.

14. The method according to claim 12, wherein the biometric sensors are configured for measuring at least one of the following conditions: heart rate, blood oxygen level, blood glucose level, electrocardiogram, and respiration rate.

15. The method according to claim 12, wherein the step of displaying or broadcasting comprises sending an audible alert signal through a speaker connected to the receiver when the threshold has been exceeded.

16. The method according to claim 12, wherein the portable monitor is embodied in a mobile telephone.

17. The method according to claim 12, wherein the remote receiver is embodied in a personal computer.

18. The method according to claim 12, wherein the transmitter is an RFID tag and the receiver is an RFID reader.

19. The method according to claim 18, wherein the microprocessor is programmed to cause the second transmitter to send an alert to the remote receiver when the RFID tag moves out of range of the RFID reader and when the sensors are removed from the patient.

20. The system according to claim 12, wherein at least one of the biometric sensors is formed from graphene.
